(19) United States
(12) Patent Application Publication
(10) Pub. No.: US 2018/0064770 A1
(43) Pub. Date: Mar. 8, 2018

(54) Composite with the Function of Regulating Blood Glucose

(71) Applicant: Ya Wen Cheng, Taipei City (TW)

(72) Inventor: Ya Wen Cheng, Taipei City (TW)

(21) Appl. No.: 15/255,532

(22) Filed: Sep. 2, 2016

(51) Int. Cl. A61K 36/06; A61K 31/315; A61K 36/54; A61K 36/63; A61K 36/42; A61K 33/24; A61K 9/20

(52) U.S. Cl. CPC A61K 36/06; A61K 31/315; A61K 36/54; A61K 9/2013; A61K 36/42; A61K 33/24; A61K 9/2018; A61K 36/63

(57) ABSTRACT

A composite with the function of regulating blood glucose is formed into a tablet by clustering and mixing bitter gourd extract, olive extract, cinnamon extract, zinc gluconate, and chromium yeast. Animal experiment results clearly show that diabetic patients have significant improvements by taking a blood glucose regulating tablet with the compound recipe of the composite.

### BACKGROUND OF THE INVENTION

### Field of the Invention

The present invention relates to a composite with the function of regulating blood glucose, more particularly to the composite that provides a significant improvement on the fasting blood glucose level of diabetic patients.

### Description of the Prior Art

In general, patients of Type 2 diabetes (primarily type 2 DM) have are obese persons with a fat midsection figure similar to those with a high Body Mass Index (BMI). People living in a city have a significant higher prevalence than people living in countryside, and this fact shows that diabetes is a disease of urbanization and industrialization.

In addition to the symptoms of too-high blood glucose and urine glucose, diabetes also may cause some complications such as retinopathy, nephropathy, nervous disorder, and disorders of brain and circulation. Therefore, how to control the blood glucose and regulate the blood glucose to a certain level to prevent the occurrence of these complications is one of the major principles of diabetes control. Up to now, there are three major methods for the treatment of diabetes, respectively: (1) Diet treatment; (2) Exercise treatment; and (3) Medication. For diet treatment, approximately 50% or more of the diabetes patients improve the condition by good diet. While using the medication method, diet treatment is also very important. If no appropriate diet treatment is cooperated, medicines will not be able to maximize their functions. Therefore, how to control the blood glucose level through a good diet treatment is a basic important subject of diabetic patients. In the foreseeing future, diabetic patients will rise year by year, so that finding a way of improving the living quality and health of the patient by health food or developing other Complementary and Alternative medicine (CAM) for regulating blood glucose are common goals of the related industry.

### SUMMARY OF THE INVENTION

Therefore, it is a primary objective of the present invention to provide a composite with the function of regulating blood glucose to overcome the problems of having an increasingly higher number of diabetic patients and break through the issue of complications.

The technical measures adopted by the present invention to overcome the aforementioned problems comprises: a tablet containing a bitter gourd extract, an olive extract, a cinnamon extract, a zinc gluconate, and a chromium yeast as active ingredients; an excipient containing a sugar alcohol or a disaccharide, and a lubricant selected from one or more items of the group consisting of a curing oil, a stearate, and a sucrose fatty acid ester, and the tablet in form of powder is formed by weighing the bitter gourd extract, olive extract, cinnamon extract, zinc gluconate, and chromium yeast precisely according to a predetermined proportion, and then sieving, and mixing the powders to form a solid-state table, wherein the excipient containing a sugar alcohol or disaccharide selected from one or more items of the group consisting of lactose, erythritol (sugar alcohol) and honeydew; wherein the tablet contains 45% of the bitter gourd extract, 12% of the olive extract, 10% of the cinnamon extract, 0.3% of the zinc gluconate, 0.18% of the chromium yeast, 0.003% of the lubricant, and 32.517% of the excipient.

The composite with the function of regulating blood glucose in accordance with the present invention has the following effect. Animal experiment results clearly show that diabetic patients have significant improvements by taking a blood glucose regulating tablet with the compound recipe of the composite.

### BRIEF DESCRIPTION OF THE DRAWINGS

FIG. 1 shows a flow chart of manufacturing a composite with the function of regulating blood glucose in accordance with the present invention;

FIG. 2 is a histogram showing a change of the blood glucose level in different groups of mice after two weeks of streptozotocin induction during an experiment in accordance with the present invention;

FIG. 3 is a histogram showing a change of the fasting blood glucose level in different groups of mice after the tablets with the compound recipe have been supplemented for two consecutive weeks in a diabetic mouse experiment in accordance with the present invention;

FIG. 4 is a histogram showing a change of the fasting blood glucose level in different groups of mice after the tables with the compound recipe have been supplemented for four consecutive weeks during a diabetic mouse experiment in accordance with the present invention; and

FIG. 5 is histogram showing a change of body weight after the tables with the compound recipe have been supplemented for four consecutive weeks during a diabetic mouse experiment in accordance with the present invention.

### DETAILED DESCRIPTION OF THE PREFERRED EMBODIMENTS

The above and other objects, features and advantages of this disclosure will become apparent from the following detailed description taken with the accompanying drawings.

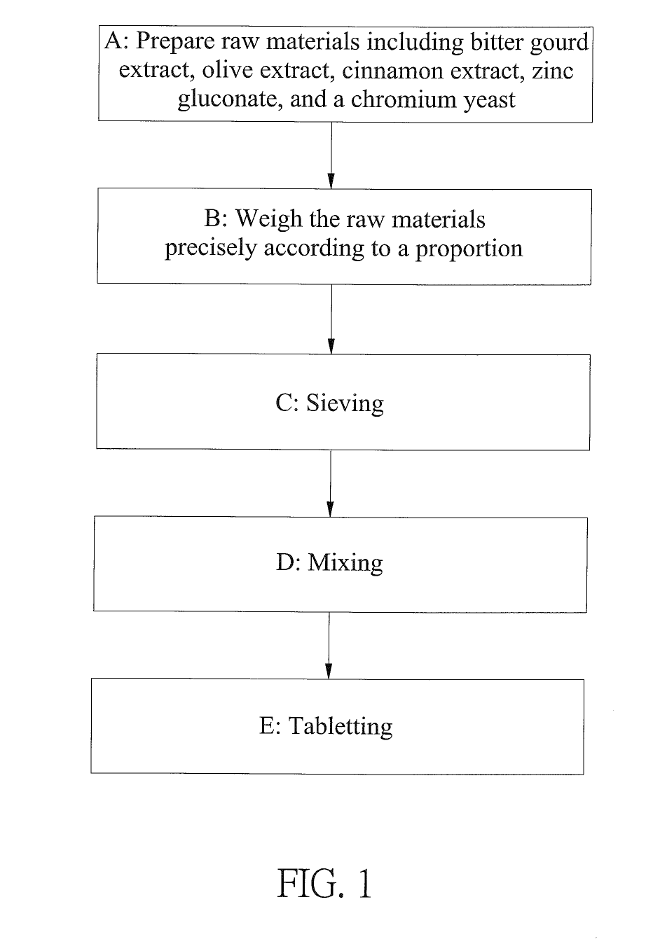

With reference to FIGS. 1 to 5 for a composite with the function of regulating blood glucose in accordance with a preferred embodiment of the present invention, the composite comprises: a tablet, containing a bitter gourd extract, an olive extract, a cinnamon extract, a zinc gluconate, and a chromium yeast as active ingredients; an excipient containing sugar alcohol or disaccharide, and a lubricant containing one or more items selected from the group consisting of a curing oil, a stearate, and a sucrose fatty acid ester.

The tablet is formed into a solid table by weighing the bitter gourd extract, the olive extract, the cinnamon extract, the zinc gluconate, and the chromium yeast which are in a powder form according to a proportion precisely and sieving, mixing, and tabletting the powders. Wherein, the excipient containing sugar alcohol or disaccharide is selected from one or more items of the group consisting of lactose, erythritol (sugar alcohol) and mannitol. Wherein, the tablet contains 45% of the bitter gourd extract, 12% of the olive extract, 10% of the cinnamon extract, 0.3% of the zinc gluconate, 0.18% of the chromium yeast, 0.003% of the lubricant, and 32.517% of the excipient.

The present invention passes the following efficacy evaluation index and animal experiment projects:

(1) Physiological Value Measurement

(a) Body Weight: During the experiment, the body weight of the mouse is measured periodically, and the body weights measured at the beginning of the experiment and measured at the end of the experiment are compared.

(b) Change of Water Intake and Feed Intake: The daily water intake and feed intake are recorded.

(2) Measurement of Fasting Blood Glucose Level:

After the hyperglyceric animals are supplemented with the composite for four weeks, blood samples are taken to measure overnight fasting blood glucose level. At present, one of the common testing methods is to use a blood glucose meter (OneTouch® UltraEasy™ of Johnson & Johnson™) which is primarily based on the principle of utilizing the electrochemical process of electric current to measure the blood glucose in a testee's blood.

All numerical values of the statistical analysis of the present invention are represented by Mean±SEM, and the number of mice of each group is at least seven. The SAS (Statistical Analysis System) computer statistical software package is used to perform the one way Analysis of variance (ANOVA), and the Duncan's test is used to test and determine whether or not there is a difference between different processes, and P<0.05 indicates a statistical significance.

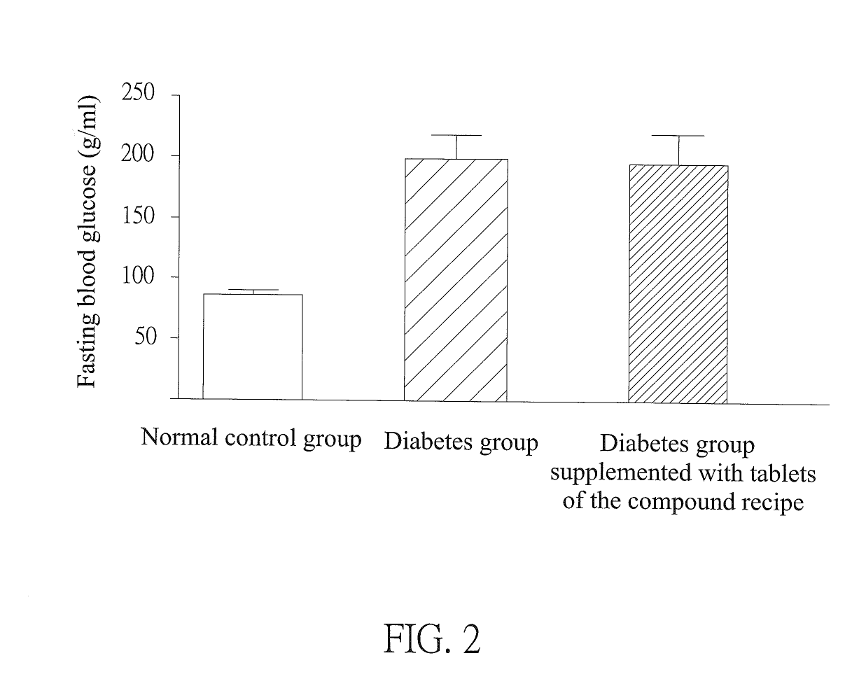

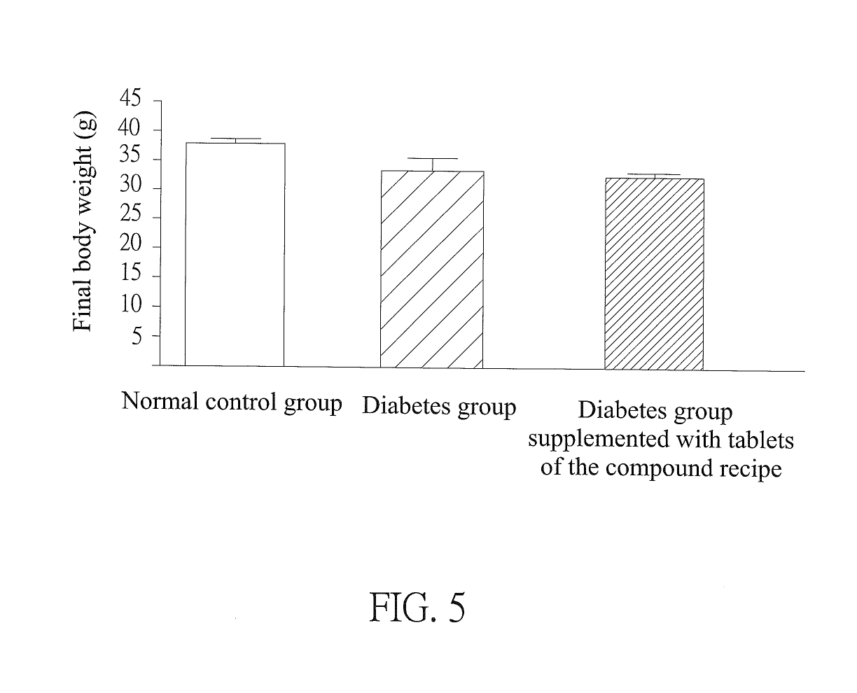

With reference to FIG. 2 for a change of blood glucose level of different groups of mice after two weeks of streptozotocin induction, international journals and literature report that an intraperitoneal injection of (150 mg/kg BW) is performed weekly, and a total of two injection take place in the streptozotocin induction of diabetic mice. After the streptozotocin has been given for the second time, all animals are fasted for 16 hours, and then the fasting blood glucose level is measured, and the blood glucose values measured in this time are used as a basis for dividing the groups. The fasting blood glucose results of a normal control group, a diabetes group with supplement of double dose, a group supplemented with tablets of the compound recipe after the induction has taken place for two weeks, the fasting blood glucose levels are equal to 87±3, 199±20, and 195±24 respectively (as shown in FIG. 5). The diabetes group and the diabetes group supplemented with double dose (recommended dosage), and the group supplemented with tablets of the compound recipe are obviously higher than the normal control group (P<0.05), and no significance exists between two groups and the means are greater than 180 mg/dL (which is equivalent to 10 mmol/L), and these results show that the model of using streptozotocin to induce diabetes has been established successfully.

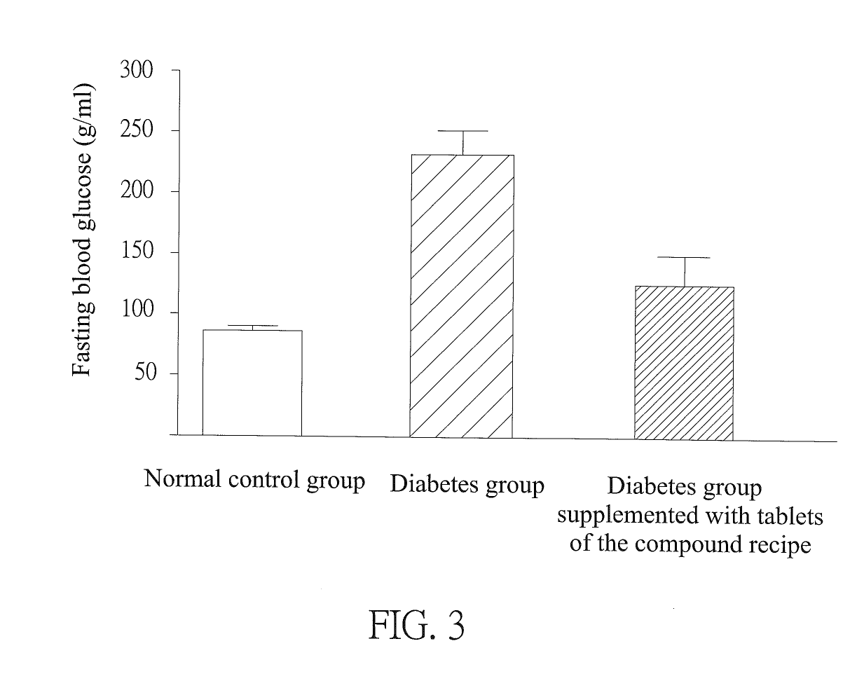

With reference to FIG. 3 for a histogram showing a change of the fasting blood glucose level in different groups of mice after the tablets with the compound recipe have been supplemented for two consecutive weeks in a diabetic mouse experiment in accordance with the present invention, after the diabetes model is induced successfully, the tablets with the compound recipe have been taken for two weeks, and then the fasting blood glucose is monitored and evaluated, and all animals are fasted for 16 hours, and then the fasting blood glucose level is measured. The fasting blood glucose levels of different groups including the normal control group, the diabetes group, the diabetes group supplemented with double dose (recommended dosage), and the group supplemented with tablets of the compound recipe are 105±4, 208±33, and 132±11 (mg/dL) respectively. The fasting blood glucose of the diabetes group is significantly higher than the normal control group by 1.98 times (P=0.0010). The fasting blood glucose levels of the diabetes groups supplemented with double dose (recommended dosage) and the group supplemented with tablets of the compound recipe are lower than the diabetes group by 36.51% (P=0.0108). Therefore, the compound recipe of double dose taken for two weeks has significant improvement on the fasting blood glucose level of the diabetic mouse.

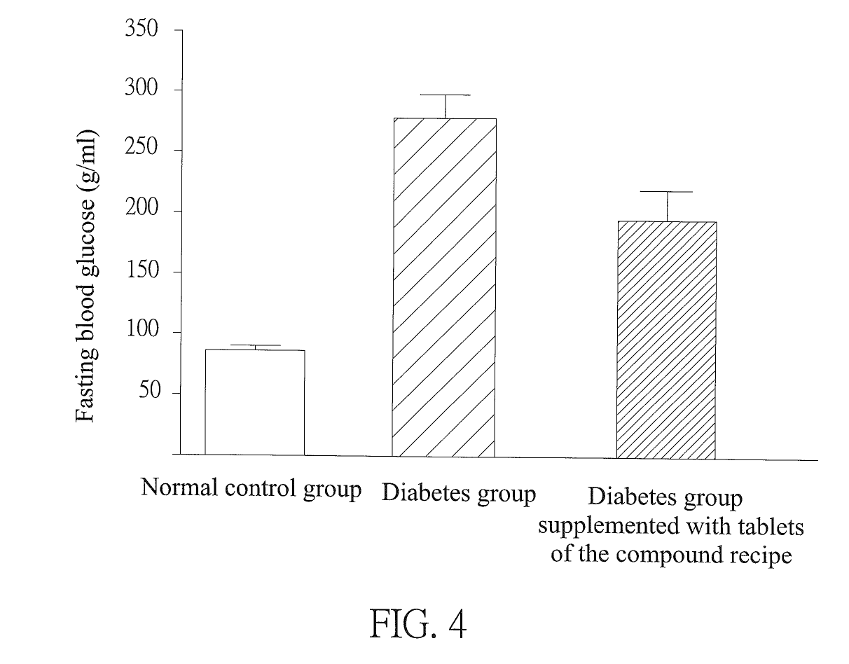

With reference to FIG. 4 for a histogram showing a change of the fasting blood glucose level in different groups of mice after the tables with the compound recipe have been supplemented for four consecutive weeks during a diabetic mouse experiment, after the diabetes model is induced successfully, the tablets with the compound recipe are taken for four consecutive weeks, and the fasting blood glucose level is monitored and evaluated, and all animals are fasted for 16 hours, and the fasting blood glucose level is measured. The fasting blood glucose levels of different groups including the normal control group, the diabetes group, the diabetes group supplemented with double dose are 105±5, 279±17, and 203±8 (mg/dL) respectively. The fasting blood glucose of the diabetes group is significantly higher than the normal control group by 2.66 times (P<0.0001). The fasting blood glucose levels of the diabetes group supplemented with double dose (recommended dosage) and the group supplemented with tablets of the compound recipe are significantly lower than the normal control group by 27.08% (P=0.0003). These results shown that the tablets of compound recipe with double dose supplemented for four consecutive weeks has a significant improvement on the fasting blood glucose level of the diabetic mouse.

With reference to FIG. 5 for a histogram showing a change of the fasting blood glucose level in different groups of mice after the tables with the compound recipe have been supplemented for four consecutive weeks during a diabetic mouse experiment, after the diabetes model is induced successfully, the tablets with the compound recipe are taken for four consecutive weeks. The body weight of the normal control group, the diabetes group, and the group of diabetes supplemented with double dose (recommended dosage) are measured at the end of the experiment to learn whether or not the tablets of the compound recipe affects the drop of body weight induced by diabetes. The body weights of the normal control group, the diabetes group, and the diabetes group supplemented with double dose (recommended dosage) measured at the end of the experiments are 37.7±1.0, 33.9±1.3, and 33.4±0.3 (g) respectively, wherein the body weight of the DM group is significantly less than the normal control group (P<0.05), and this result complies with the clinical symptom of diabetes (drop of body weight). After the double dose is applied, the body weights of the diabetes group supplemented with double dose (recommended dosage), and the group supplemented with tablets of the compound recipe are increased slightly (compared with the diabetes group), but there is no statistical significance.

The results of the present invention indicate the following effects:

Composite with the function of regulating Blood Glucose:

According to the statistics of the aforementioned fasting blood glucose levels measured before and after the experiment shows a significance (P<0.05), so that the tablet with the compound recipe is preliminarily decided to have the effect of regulating blood glucose.

The composite with the function of regulating blood glucose in accordance with the present invention has the following effect. Animal experiment results clearly show that diabetic patients have significant improvements by taking a blood glucose regulating tablet with the compound recipe of the composite.

While various embodiments in accordance with the present invention have been shown and described, it is clear to those skilled in the art that further embodiments may be made without departing from the scope of the present invention.

What is claimed is:

1. A composite with the function of regulating blood glucose, comprising:
a tablet, containing a bitter gourd extract, an olive extract, a cinnamon extract, a zinc gluconate, and a chromium yeast as active ingredients, an excipient containing sugar alcohol or disaccharide, and a lubricant containing one or more items selected from the group consisting of curing oil, stearate and sucrose fatty acid ester.

2. The composite with the function of regulating blood glucose according to claim 1, wherein the tablet is formed into a solid table by weighing the bitter gourd extract, the olive extract, the cinnamon extract, the zinc gluconate, and the chromium yeast which are in a powder form according to a proportion precisely and sieving, mixing, and tabletting the powders.

3. The composite with the function of regulating blood glucose according to claim 1, wherein the excipient containing sugar alcohol or disaccharide is selected from one or more items of the group consisting of lactose, erythritol (sugar alcohol) and mannitol.

4. The composite with the function of regulating blood glucose according to claim 1, wherein the tablet contains 45% of the bitter gourd extract, 12% of the olive extract, 10% of the cinnamon extract, 0.3% of the zinc gluconate, 0.18% of the chromium yeast, 0.003% of the lubricant, and 32.517% of the excipient.
